(19) United States
(12) Patent Application Publication
(10) Pub. No.: US 2002/0198402 A1
(43) Pub. Date: Dec. 26, 2002

(54) Citric esters and a process for their preparation

(76) Inventors: Hans Bohnen, Moers (DE); Klaus Bergrath, Oberhausen (DE); Thomas Klein, Oberhausen (DE)

Correspondence Address: BIERMAN MUSERLIAN AND LUCAS, 600 THIRD AVENUE, NEW YORK, NY, 10016

(21) Appl. No.: 10/140,959

(22) Filed: May 8, 2002

(30) Foreign Application Priority Data
May 8, 2001 (DE) 101 22 145.2

(51) Int. Cl. C07C 69/66

(52) U.S. Cl. 560/179

(57) ABSTRACT

Mixtures of critic acid esters useful as plasticizers and a process of producing the same.

The present invention relates to a mixture of citric esters, to a process for their preparation, and also to their use as plasticizers.

The ester mixtures of the invention have very low volatility and excellent plasticizing capability for thermoplastics, and therefore have excellent suitability for applications in the plastics sector.

Plasticizers are widely used in plastics, in coating compositions, and in sealing compounds, and also in items made from various forms of rubber. They enter into physical interaction, preferably via their capability for solvating and swelling, with thermoplastic high polymers but do not react chemically. The result is a homogeneous system with a thermoplastic range which has been shifted to lower temperatures when compared with that of the original polymer. The addition of plasticizers results, inter alia, in a material whose mechanical properties are improved over those of the untreated starting material. Capability for dimensional change is improved, for example, as are elasticity and strength, and hardness is reduced. An important property of plasticizers is therefore their capability to plasticize plastics. To achieve this type of advantageous action, plasticizers have to have good compatibility with the plastic so that it becomes possible to incorporate even large amounts into the plastic composition.

In order that plasticizers have the widest possible scope of application, a large number of generally applicable criteria have to be complied with. Ideally, they should be odorless, colorless, lightfast, and heat-resistant. In addition, it is desirable for them to be water-resistant, and to have low flammability, low combustibility, and low volatility, and to have minimal tendency to migrate out of the plastic composition when used as specified. Particular importance is attached especially to low volatility of plasticizers, and this is significant both during incorporation into the plastic composition and during practical use of the moldings. In addition, for applications in the food and drink sector and in the medical sector the plasticizers provided have to be non-hazardous to health. Finally, the preparation of the plasticizers should be simple, both with respect to the apparatus needed and with respect to the steps required in the process, and the formation of non-recyclable by-products and pollutant wastes should be avoided in order to comply with environmental requirements.

The excellent plasticizing properties of certain phthalates give them wide application as additives for thermoplastics, in particular PVC. However, they are continually faced with health concerns, and this militates against their universal use. For example, they cannot be used in connection with food or drink, e.g. as a material in packaging, nor in other products whose use is subject to particular precautions for reasons of preventive health care. They include items in day-to-day use, such as household products and child-care products, including toys, and also products used in the medical sector. The plasticizers used for thermoplastic auxiliary or finished products intended for these specific application sectors are therefore not phthalates but citric esters, which are toxicologically non-hazardous.

The use of citric esters based on a single alcohol, for example based on n-butanol or 2-ethylhexanol, is known (Gächter/Müller, Kunststoffadditive [Plastics Additives], 3rd Edition 1990, Carl Hanser-Verlag, pp. 417-418). These esters frequently also undergo further derivatization, by acylation of the free hydroxy group of the citric acid via reaction with a carboxylic acid or carboxylic acid derivative, for example with the anhydride. An acylation process of this type is known by way of example from DE-B-1 099 523. DE-A1 -35 20 750 also discloses citric esters of this type or their acylated forms in which various alcohols have been bonded to the citric acid, for example tri-n-(hexyl/octyl/decyl) acetyl citrate. The known mixed esters are used for producing medical items.

Citric esters have to have low volatility, in order that only very small losses of product occur during processing, and very little off-gas pollution of the air in the working environment. However, on the other hand they have to have good compatibility with the plastic so that even large amounts can be incorporated into the thermoplastic and that no bleed-out of the plasticizers from the thermoplastic occurs even over prolonged periods of use. The citric esters known to date are not entirely satisfactory in meeting both of these requirements simultaneously.

An object of the invention was therefore to provide citric esters which have both low volatility and excellent compatibility with a thermoplastic, so that large amounts of these can be incorporated into the thermoplastic.

This object is achieved by way of a mixture of citric esters comprising, based on the total weight of the ester, from 5 to 40% by weight of tri-n-butyl citrate, from 59 to 77% by weight of the compound of the formula (I)

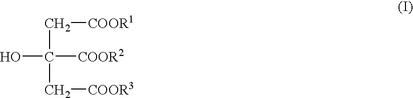

where R¹. R² and R³ are a straight-chain or branched alkyl radical having from 4 to 10 carbon atoms, with the proviso that at least one of the radicals R¹, R², or R³ is n-butyl and the two other radicals are identical or different but all of the radicals R¹, R², and R³ are not simultaneously n-butyl, and from 1 to 18% by weight of the compound of the general formula (I), with the proviso that R¹, R², and R³ are identical but not n-butyl.

Surprisingly, the citric ester mixtures of the invention have excellent low volatility performance together with exceptional compatibility with the thermoplastic. Large amounts of these can therefore be incorporated into plastics, and they are therefore particularly suitable for producing moldings which have to be highly flexible, for example for flexible hoses and containers used in medical technology.

The alcohol component used besides n-butanol is isobutanol or an aliphatic monoalcohol having from 5 to 10 carbon atoms, from any desired source. Oxo alcohols are in particular used since they have low volatility, these being alcohols which have been prepared by an oxo synthesis, i.e. by reacting monoolefins with carbon monoxide and hydrogen. It is preferable to use n-pentanol, n-heptanol, 2-ethylhexanol, n-octanol, n-decanol, or isodecanols, e.g. 2-propylheptanol.

The makeup of the ester mixture of the invention, based on the total weight of the ester, is preferably from 8 to 30% by weight of tri-n-butyl citrate, from 67 to 75% by weight of the compound of the general formula (I), with the proviso that at least one of the radicals R¹, R², or R³ is n-butyl and the other two radicals are identical or different, but all of the radicals R¹, R², and R³ are not simultaneously n-butyl, and from 3 to 17% by weight of the compound of the general formula (I) with the proviso that R¹, R², and R³ are identical or different but are not n-butyl.

An ester mixture which has proven particularly suitable is one in which the second alcohol component is 2-ethylhexanol and which, based on the total weight of the ester, comprises 26% by weight of tri-n-butyl citrate, 45% by weight of di-n-butyl 2-ethylhexyl citrate, 25% by weight of n-butyl di-2-ethylhexyl citrate, and 4% by weight of tri-2-ethylhexyl citrate.

Volatility is measured with the aid of a Brabender high-speed moisture tester with rotating pan. In addition, the weight loss is determined from a liquid specimen when the specimen is subjected to heat treatment at 150° C. for a period of 2 hours.

Plasticizing capability is determined via the critical solubility temperature to DIN 53408, the PVC grade used for the test being S-1067.

Surprisingly, the ester mixtures of the invention exhibit markedly lower volatility than would be expected from routine interpolation between the values measured on tri-n-butyl citrate and on the compound of the formula (I) where R¹, R², and R³ are identical or different but not n-butyl. The critical solubility temperature plot is found to be almost linear, with an increasing temperature rise in the direction of the compound where R¹, R² and R³ are identical or different but are not n-butyl. The ester mixtures of the invention therefore achieve the object of providing citric esters which have improved volatility performance while retaining very good plasticizing capability.

The ester mixture of the invention is prepared by reaction of citric acid with the alcohol mixture comprising n-butanol. The reaction is carried out with excess alcohol mixture in the presence of catalysts, in order to achieve the fullest possible conversion of the acid in an acceptable time. The molar ratio of citric acid to alcohol mixture is generally greater than 1:3.6, this molar ratio being based on the hydroxy groups present in the alcohol mixture. For high conversion and the high product yield associated therewith it is advantageous not only to use an excess of alcohol but also continuously to remove the water formed in the reaction. The catalysts which have proved successful are acids, which may be present in solution or suspension in the reaction mixture. The ester synthesis is followed by the removal of the catalyst and washing of the product with water. The excess alcohol is then separated off from the reaction mixture by distillation. The residue is then dried at reduced pressure and elevated temperature, where appropriate with the aid of an inert gas stream. The mixed esters of the invention are found as clear, colorless liquids in the residue from the drying process. To prepare the ester mixtures of the invention, an alcohol mixture which has an n-butanol content of from 50 to 90 mol % and a content of from 10 to 50 mol % of the second monoalcohol component R′-OH is subjected to esterification. The second monoalcohol component may either be a single compound, where R′ is identical with R¹. identical with R², or identical with R³, and R¹, R³ and R are not simultaneously n-butyl. The second alcohol component may also be a mixture made from two different monoalcohols other than n-butanol. A particularly successful method has proven to be the use of an alcohol mixture which comprises from 50 to 70 mol % of n-butanol and from 30 to 50 mol % of the single monoalcohol R′-OH. The esterification in particular uses a mixture made from n-butanol and 2-ethylhexanol or made from n-butanol and isodecanol, such as 2-propylheptanol.

A successful temperature range for reacting the citric acid and the alcohol mixture has proven to be from 110 to 140° C. Lower temperatures are not excluded as long as the particular nature of the reaction partners or of the reaction conditions leads to achievement of a sufficiently high reaction rate, or only partial conversions are desired. Higher temperatures are generally avoided in order to eliminate the risk of decomposing the starting materials, by-products, and final products, and resultant contamination of the mixed ester, e.g. by substances which adversely affect color or by aconitic acid, which is formed by elimination of water from citric acid. It is possible to use a reduced pressure during the reaction, but this embodiment of the process will be restricted to specific cases.

To ensure that reaction times are economically acceptable it is necessary to increase the rate of reaction of acid and alcohol by adding a catalyst. The usual catalytically active substances are suitable for this purpose, for example titanates, sulfuric acid, formic acid, polyphosphoric acid, methanesulfonic acid, or para-toluenesulfonic acid, these being used either in the form of a mixture of various substances or as pure compound, dissolved or suspended in the reaction mixture. Preference is given to sulfuric acid, methanesulfonic acid, or para-toluenesulfonic acid, these being commercially available at low cost and capable of easy removal from the reaction mixture. The amount of catalyst used may vary over a wide range. It is possible to use either 0.01 % by weight or else 5% by weight of catalyst, for example, based on the reaction mixture. However, since larger amounts of catalyst give few advantages, the catalyst concentration is usually from 0.01 to 1.0% by weight, preferably from 0.01 to 0.5% by weight, based in each case on the reaction mixture.

Removal of the water of reaction from the reaction mixture is required in order to shift the esterification equilibrium in the direction of the desired product, and this takes place with the aid of azeotrope-formers (entrainers). The substances selected for this purpose are usually organic solvents which with water form mixtures boiling within the reaction temperature range, at from 110 to 140° C. Examples of suitable entrainers are hexane, cyclohexane, toluene, and the isomeric xylenes, cyclohexane being preferred. The amount of entrainer required for complete removal of the water may be determined by a simple method from the amount of water formed, calculated on the basis of the stoichiometry of the esterification reaction, and the makeup of the binary azeotrope. It has proven successful to use an excess of entrainer, the proportion advantageously being from 50 to 200% by weight above the amount calculated from theory. Careful selection of the amount of entrainer within this range can permit the esterification temperature to be set to the desired value within the region from 110 to 140° C. The progress of the reaction can be followed by a simple method via collection and removal of the distilled entrainer/water mixture. The entrainer separated out from the azeotrope may be returned directly to the reaction.

Following the esterification reaction, the acidic catalyst is neutralized by adding alkaline reagents, for example by adding an aqueous solution of sodium hydroxide, sodium carbonate, or sodium hydrogen carbonate, by mixing the crude ester intimately with the alkaline solution. The crude ester obtained after phase separation is washed with water to remove the final traces of alkali. Excess alcohol, and also entrainer residues, are then removed by distillation, and the distillation residue is dried at reduced pressure and elevated temperature, where appropriate by introducing an inert gas.

The triesters obtained via reaction of citric acid and the alcohol mixture still contain a free hydroxy group. It is known that the compatibility of citric triesters in thermoplastics can be increased if the hydroxy group is derivatized to give an acyl group. The introduction of the acyl group also increases the thermal stability of the citric esters, since the free hydroxy group can be eliminated from the citric esters on exposure to heat, forming water and giving unsaturated compounds which are undesirable contaminants in the ester product.

The invention therefore also provides a mixture of acylated citric esters in which the citric esters present in the ester mixture of the invention are present in acylated form, where the acyl group may be linear or branched and contains from 2 to 5 carbon atoms.

To acylate the hydroxy-containing esters present in the ester mixture of the invention, use is made of a linear or branched carboxylic acid having from 2 to 5 carbon atoms in the molecule or a derivative of the same, preferably the corresponding anhydride or the acid chloride. Acetic anhydride, propionic anhydride, or butyric anhydride have proven to be particularly successful acylating agents.

If the acylated compounds are the desired products, their preparation advantageously begins with the crude ester obtained after neutralization, water wash, and distillation to remove the excess alcohol and any entrainer residues.

The free hydroxy group is esterified using the carboxylic acid or the derivative of the carboxylic acid, preferably the anhydride, an excess of which is used, based on the triester. It is advantageous to use from 1.2 to 1.8 mol, preferably from 1.3 to 1.5 mol, of a monocarboxylic acid, or the same amount of the derivative of the acid, per mole of triester. The reaction temperature is advantageously kept to not more than 110° C., the particular temperature selected on any occasion depending on the reactivity of the ester and of the acylating agent. The temperature range from 60 to 80° C. is preferred. Again, a catalyst is generally added to the reaction mixture, and the catalyst used in the acylating stage are those which have also proven successful in the esterification stage. The amount of catalyst here is generally from 0.01 to 5% by weight, preferably from 0.01 to 1.0% by weight, and particularly preferably from 0.01 to 0.5% by weight, based in each case on the reaction mixture.

Once acylation has been completed, excess acylating agent and other volatile compounds, such as the carboxylic acid released from the acylating agent if an anhydride is used as acylating agent, are then removed from the crude mixture by distillation. Distillation is followed by neutralization of the crude acylation mixture, carried out using a method similar to that for the neutralization which followed the esterification reaction. After washing with water, the product is dried by conventional methods to remove final traces of moisture. Examples of methods for removing the residual water are to use slightly elevated temperatures at reduced pressure or to pass a stream of an inert gas, such as nitrogen, through the residue.

The examples below describe the process of the invention in more detail, but the novel procedure is not restricted to this one embodiment.

### EXAMPLES

### Example 1

### Preparation of 70/30 Citrate

1681.0 9 of citric acid monohydrate (8 mol), 1491.8 g of n-butanol (20.2 mol), 1118.0 g of 2-ethylhexanol (8.6 mol), and also 9.0 g of methanesulfonic acid (0.1 mol), and 252 g of cyclohexane were charged to a three-necked flask equipped with stirrer, internal thermometer, and water separator and stirred until a homogeneous solution was produced. The reaction mixture was then heated to 120° C., and the water produced in the reaction was removed from the reaction mixture over a period of 9 hours at this temperature. Once the reaction had ended, the mixture was neutralized by adding 201.8 g of water and 162.9 g of a 3% strength aqueous NaOH solution. After phase separation, the organic product phase was washed with water. In order to separate off the excess alcohol content, the crude ester was subjected to steam distillation, and then dried at 120° C. and at a pressure of about 100 Pa in a stream of nitrogen to remove residual water. The resultant ester mixture had the following makeup, determined by gas chromatography.

GC makeup of ester mixture (% by weight)

| | |
|---|---|
| Tri-n-butyl citrate | 25.6 |
| Di-n-butyl 2-ethylhexyl citrate | 44.8 |
| n-Butyl di-2-ethylhexyl citrate | 24.5 |
| Tri-2-ethylhexyl citrate | 4.3 |
| Remainder | 0.8 |

### Example 2

### Preparation of 50/50 Citrate

2101.4 g of citric acid monohydrate (10 mol), 1334.2 g of n-butanol (18 mol), 2340.0 g of 2-ethylhexanol (18 mol), and also 10.0 g of methanesulfonic acid (0.1 mol), and 200 g of cyclohexane were charged to a three-necked flask equipped with stirrer, internal thermometer, and water separator and stirred until a homogeneous solution was produced. The reaction mixture was then heated to 120° C., and the water produced in the reaction was removed from the reaction mixture over a period of 15 hours at this temperature. Once the reaction had ended, the mixture was neutralized by adding 160 g of water and 55.2 9 of a 20% strength aqueous NaOH solution. After phase separation, the organic product phase was washed with water. In order to separate off the excess alcohol content, the crude ester was subjected to steam distillation, and then dried at 120° C. and at a pressure of about 100 Pa in a stream of nitrogen to remove residual water. The resultant ester mixture had the following makeup, determined by gas chromatography.

GC makeup of ester mixture (% by weight)

| | |
|---|---|
| Tri-n-butyl citrate | 8.1 |
| Di-n-butyl 2-ethylhexyl citrate | 32.8 |
| n-Butyl di-2-ethylhexyl citrate | 42.0 |
| Tri-2-ethylhexyl citrate | 16.9 |
| Remainder | 0.2 |

The volatility values for the resultant ester mixtures were determined in the Brabender high-speed moisture tester (150° C. /2 hours), and the critical solubility temperature to DIN 53408 was determined using PVC of grade S 1067. For comparative purposes, pure tri-n-butyl citrate and tri-2-ethylhexyl citrate were also tested. The table below shows the results from the tests.

TABLE 1
Volatility and critical solubility temperature of citric esters

| | | Volatility | Critical solubility |
|---|---|---|---|
| Number | Citric ester | (%) | temperature (° C.) |
| 1 | Tri-n-butyl citrate | 2.48 | 108 |
| 2 | 70/30 citrate (Example 1) | 0.95 | 127 |
| 3 | 50/50 citrate (Example 2) | 0.69 | 138 |
| 4 | Tri-2-ethylhexyl citrate | 0.23 | 177 |

As can be seen from Table 1, the volatility values measured on the ester mixtures of the invention (Examples 2 and 3) are markedly below the volatility values which would be expected from routine interpolation between the volatility values measured for tri-n-butyl citrate and tri-2-ethylhexyl citrate. In comparison with pure tri-n-butyl citrate, which on the one hand has good plasticizing capability but on the other hand has high volatility and suffers weight loss of 2.5% by weight in the Brabender high-speed test, the volatility value can be lowered to about 1.0% by weight by using an ester mixture which comprises about 26% by weight of tri-n-butyl citrate, 4% by weight of tri-2-ethylhexyl citrate, and 70% by weight of mixed esters. The rise here in the critical solubility temperature is merely from 108 to 127° C. Whereas the critical solubility temperature measure is within the scope of the interpolated rise between the marker values for pure tri-n-butyl citrate and tri-2-ethylhexyl citrate, the reduction in volatility is markedly more pronounced than would be expected from the interpolated volatility curve. On the basis of the interpolated volatility values, the type of reduction in volatility found was unexpected.

What is claimed is:

1. A mixture of citric esters comprising, based on the total weight of the ester, 5 to 40% by weight of tri-n-butyl citrate, 59 to 77% by weight of the compound of the formula 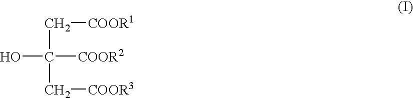
wherein R¹, R² and R³ are individually alkyl of 4 to 10 carbon atoms, with the proviso that at least one R¹, R², or R³ is n-butyl and the other two R¹, R² and R³ are not all simultaneously n-butyl, and 1 to 18% by weight of a compound of the general formula (I), with the proviso that R¹, R², and R³ are identical or different but not n-butyl.

2. The mixture of claim 1, comprising, based on the total weight of the ester, 8 to 30% by weight of tri-n-butyl citrate, 67 to 75% by weight of the compound of the formula (I), with the proviso that at least one R¹, R², or R³ is n-butyl and the other two are individual, but all of R¹, R², and R³ are not simultaneously n-butyl, and 3 to 17% by weight of the compound of the formula (I), with the proviso that R¹, R², and R³ are identical or different but are not n-butyl.

3. The mixture of claim 1, wherein R¹, R², and R³ are individually selected from the group consisting of n-pentyl, n-heptyl, 2-ethylhexyl, n-octyl, n-decyl, isodecyl and 2-propylheptyl.

4. A mixture of citric esters of claim 1, comprising, based on the total weight of the ester, 26% by weight of tri-n-butyl citrate, 45% by weight of di-n-butyl 2-ethylhexyl citrate, 25% by weight of n-butyl di-2-ethylhexyl citrate, and 4% by weight of tri-2-ethylhexyl citrate.

5. A mixture of acylated citric esters of claim 1, wherein the citric esters in the mixture contain an acyl of 2 to 5 carbon atoms.

6. The mixture of claim 5, wherein the acyl group is selected from the group consisting of acetyl, propionyl and butyryl.

7. A process for preparing mixtures of claim 1, comprising
(a) reacting citric acid with a butanol-containing alcohol mixture in the presence of catalysts, with the aid of an entrainer to remove the water formed in the reaction; and
(b) removing the catalyst, washing of the product with water, separation of the excess alcohol from the product, and drying at reduced pressure and elevated temperature.

8. The process of claim 7, wherein a mixture of n-butanol and 2-ethylhexanol is esterified with citric acid.

9. The process of claim 7, wherein a mixture of n-butanol and isodecanol is esterified with citric acid.

10. A process for preparing the mixture of acylated citric esters of claim 5 comprising
(a) reacting citric acid with a butanol-containing alcohol mixture in the presence of catalysts, using an entrainer to remove the water formed in the reaction;
(b) removing the catalyst, washing of the product with water, separating the excess alcohol from the product;
(c) acylating the free hydroxy group of the citric esters in the presence of a catalyst with monocarboxylic acid of 2 to 5 carbon atoms or a derivative of this monocarboxylic acid;
(d) removing the excess acylating agent and of its reaction products;
(e) neutralizing the catalyst used in the acylating stage, washing with water and drying at reduced pressure and elevated temperature.

11. The process of claim 10, wherein the acylating agent is selected from the group consisting of acetic anhydride, propionic anhydride and butyric anhydride in step (c).

12. The process of claim 10, wherein a mixture of n-butanol and 2-ethylhexanol is esterified with citric acid.

13. The process of claim 10, wherein a mixture of n-butanol and isodecanol is esterified with citric acid.

14. In the process of plasticizing thermoplastic resins, the improvement comprising using as the plasticizer, a mixture of claim 1.

15. In the process of plasticizing thermoplastic resins, the improvement comprising using as the plasticizer, a mixture of claim 5.

16. A plasticizer composition comprising an ester mixture of claim 1.

17. A plasticizer composition comprising an ester mixture of claim 5.
